SEQUENCE LISTING (33 sequences)

SEQ ID NO: 1 - protein, 249 aa, Thermoascus aurantiacus
MSFSKIIATAGVLASASLVAGHGFVQNIVIDGKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 2 - protein, 250 aa, Aspergillus fumigatus [sequence omitted]

SEQ ID NO: 3 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 4 - protein, 239 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 5 - protein, 258 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 6 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 7 - protein, 304 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 8 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 9 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 10 - protein, 232 aa, Myceliophthora thermophila
MKFTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIDDGEYLLRVEHIALHSASSVGGAQFYIACAQLSVTGGSGTLNTGSLVSLPGAYKATDPGILFQLYWPIPTEYINPGPAPVSC

SEQ ID NO: 11 - protein, 235 aa, Myceliophthora thermophila
MKALSLLAAASAVSAHTIFVQLEADGTRYPVSYGIRDPSYDGPITDVTSNDVACNGGPNPTTPSSDVITVTAGTTVKAIWRHTLQSGPDDVMDASHKGPTLAYLKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 12 - protein, 323 aa, Myceliophthora thermophila
MKSFALTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGEGYTGCTNCASGSTCSAVSPPYYSQCV

SEQ ID NO: 13 - protein, 310 aa, Myceliophthora thermophila
MKPFSLVALATAVSGHAIFQRVSVNGQDQGQLKGVRAPSSNSPIQNVNDANMACNANIVYHDSTIIKVPAGARVGAWWQHVIGGPQGANDPDNPIAASHKGPIQVYLAKVDNAATASPSGLRWFKVAERGLNNGVWAVDELIANNGWHYFDLPSCVAPGQYLMRVELLALHSASSPGGAQFYMGCAQIEVTGSGTNSGSDFVSFPGAYSANDPGILLSIYDSSGKPTNGGRSYPIPGPRPISCSGSGDGGNNGGGGDDNNNNNGGGNNGGGGGGSVPLYGQCGGIGYTGPTTCAQGTCKVSNEYYSQCLP

SEQ ID NO: 14 - protein, 246 aa, Myceliophthora thermophila
MKLSLFSVLATALTVEGHAIFQKVSVNGADQGSLTGLRAPNNNNPVQDVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPPSTVSFPGAYSASDPGILINIYGATGQPDNNGQPYTAPGPAPISC

SEQ ID NO: 15 - protein, 354 aa, Thermoascus aurantiacus
MSFSKIAAITGAITYASLAAAHGYVTGIVADGTYYGGYIVTQYPYMSTPPDVIAWSTKATDLGFVDPSSYASSDIICHKGAEPGALSAKVAAGGTVELQWTDWPESHKGPVIDYLAACNGDCSTVDKTKLEFFKIDESGLIDGSSAPGTWASDNLIANNNSWTVTIPSTIAPGNYVLRHEIIALHSAGNTNGAQNYPQCINLEVTGSGTDTPAGTLGTELYKATDPGILVNIYQTLTSYDIPGPALYTGGSSGSSGSSNTAKATTSTASSSIVTPTPVNNPTVTQTAVVDVTQTVSQNAAVATTTPASTAVATAVPTGTTFSFDSMTSDEFVSLMRATVNWLLSNKKHARDLSY

SEQ ID NO: 16 - protein, 322 aa, Penicillium pinophilum
MPSTKVAALSAVLALASTVAGHGFVQNIVIDGKSYSGYLVNQFPYESNPPAVIGWATTATDLGFVAPSEYTNADIICHKNATPGALSAPVAAGGTVELQWTTWPDSHHGPVISYLANCNGNCSTVDKTKLDFVKIDQGGLIDDTTPPGTWASDKLIAANNSWTVTIPSTIAPGNYVLRHEIIALHSAGNADGAQNYPQCINLEITGSGTAAPSGTAGEKLYTSTDPGILVNIYQSLSTYVIPGPTLWSGAANGAVATGSATAVATTATASATATPTTLVTSVAPASSTFATAVVTTVAPAVTDVVTVTDVVTVTTVITTTVL

SEQ ID NO: 17 - protein, 444 aa, Thermoascus sp.
MLSFASAKSAVLTTLLLLGSAQAHTLMTTLFVDGVNQGDGVCIRMNNNGSTANTYIQPVTSKDIACGIQGEIGAARVCPAKASSTLTFQFREQPSNPNSAPLDPSHKGPAAVYLKKVDSAIASNNAAGDGWFKIWESVYDESTGKWGTTKMIENNGHISVKVPDDIEGGYYLARTELLALHAANEGDPQFYVGCAQLFIDSAGTAKPPTVSIGEGTYDLSMPAMTYNIYQTPLALPYPMYGPPVYTPGSGSGSGSGSGSASATRSSAIPTATAVTDCSSEEDREDSVMATGVPVARSTLRTWVDRLSWHGKARENVKPAARRSALVQTEGLKPEGCIFVNGNWCGFEVPDYNDAESCWAASDNCWKQSDSCWNQTQPTGYNNCQIWQDQKCKPIQDSCSQSNPTGPPNKGKDITPTWPPLEGSMKTFTKRTVSYRDWIMKRKGA

SEQ ID NO: 18 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 19 - protein, 223 aa, Thielavia terrestris
MKLSSQLAALTLAAASVSGHYIFEQIAHGGTKFPPYEYIRRNTNYNSPVTSLSSNDLRCNVGGETAGNTTVLDVKAGDSFTFYSDVAVYHQGPISLYMSKAPGSVVDYDGSGDWFKIHDWGPTFSNGQASWPLRDNYQYNIPTCIPNGEYLLRIQSLAIHNPGATPQFYISCAQVRVSGGGSASPSPTAKIPGAFKATDPGYTANIYNNFHSYTVPGPAVFQC

SEQ ID NO: 20 - protein, 246 aa, Thielavia terrestris
MKFSLVSLLAYGLSVEAHSIFQRVSVNGQDQGLLTGLRAPSNNNPVQDVNSQNMICGQSGSKSQTVINVKAGDRIGSLWQHVIGGAQFSGDPDNPIAHSHKGPVMAYLAKVDNAASASQTGLKWFKIWQDGFDTSSKTWGVDNLIKNNGWVYFHLPQCLAPGQYLLRVEVLALHSAYQQGQAQFYQSCAQINVSGSGSFSPSQTVSIPGVYSATDPSILINIYGSTGQPDNGGKAYNPPGPAPISC

SEQ ID NO: 21 - protein, 334 aa, Thielavia terrestris
MRTTFAAALAAFAAQEVAGHAIFQQLWHGSSCVRMPLSNSPVTNVGSRDMICNAGTRPVSGKCPVKAGGTVTVEMHQQPGDRSCNNEAIGGAHWGPVQVYLSKVEDASTADGSTGWFKIFADTWSKKAGSSVGDDDNWGTRDLNACCGKMQVKIPADIPSGDYLLRAEALALHTAGQVGGAQFYMSCYQITVSGGGSASPATVKFPGAYSANDPGIHINIHAAVSNYVAPGPAVYSGGTTKVAGSGCQGCENTCKVGSSPTATAPSGKSGAGSDGGAGTDGGSSSSSPDTGSACSVQAYGQCGGNGYSGCTQCAPGYTCKAVSPPYYSQCAPSS

SEQ ID NO: 22 - protein, 227 aa, Thielavia terrestris
MKLSVAIAVLASALAEAHYTFPSIGNTADWQYVRITTNYQSNGPVTDVTSDQIRCYERNPGTGAQGIYNVTAGQTINYNAKASISHPGPMSFYIAKVPAGQTAATWDGKGAVWTKIYQDMPKFGSSLTWPTMGAKSVPVTIPRCLQNGDYLLRAEHIALHSASSVGGAQFYLSCAQLTVSGGSGTWNPKNRVSFPGAYKATDPGILINIYYPVPTSYSPPGPPAETC

SEQ ID NO: 23 - protein, 368 aa, Thielavia terrestris
MPSFASKTLLSTLAGAASVAAHGHVSNIVINGVSYQGYDPTSFPYMQNPPIVVGWTAADTDNGFVAPDAFASGDIICHKNATNAKGHAVVAAGDKIFIQWNTWPESHHGPVIDYLASCGSASCETVDKTKLEFFKIDEVGLVDGSSAPGVWGSDQLIANNNSWLVEIPPTIAPGNYVLRHEIIALHSAENADGAQNYPQCFNLQITGTGTATPSGVPGTSLYTPTDPGILVNIYSAPITYTVPGPALISGAVSIAQSSSAITASGTALTGSATAPAAAAATTTSTTNAAAAATSAAAAAGTSTTTTSAAAVVQTSSSSSSAPSSAAAAATTTAAASARPTGCSSGRSRKQPRRHARDMVVARGAEEAN

SEQ ID NO: 24 - protein, 330 aa, Thielavia terrestris
MPPALPQLLTTVLTALTLGSTALAHSHLAYIIVNGKLYQGFDPRPHQANYPSRVGWSTGAVDDGFVTPANYSTPDIICHIAGTSPAGHAPVRPGDRIHVQWNGWPVGHIGPVLSYLARCESDTGCTGQNKTALRWTKIDDSSPTMQNVAGAGTQGEGTPGKRWATDVLIAANNSWQVAVPAGLPTGAYVLRNEIIALHYAARKNGAQNYPLCMNLWVDASGDNSSVAATTAAVTAGGLQMDAYDARGFYKENDPGVLVNVTAALSSYVVPGPTVAAGATPVPYAQQSPSVSTAAGTPVVVTRTSETAPYTGAMTPTVAARMKGRGYDRRG

SEQ ID NO: 25 - protein, 236 aa, Thielavia terrestris
MKTFTALLAAAGLVAGHGYVDNATIGGQFYQNPAVLTFFQPDRVSRSIPGNGPVTDVTLIDLQCNANSTPAKLHATAAAGSDVILRWTLWPESHVGPVITYMARCPDTGCQDWMPGTSAVWFKIKEGGRDGTSNTWADTPLMTAPTSYTYTIPSCLKKGYYLVRHEIIALHAAYTYPGAQFYPGCHQLNVTGGGSTVPSSGLVAFPGAYKGSDPGITYDAYKAQTYQIPGPAVFTC

SEQ ID NO: 26 - protein, 250 aa, Thielavia terrestris
MALLLLAGLAILAGPAHAHGGLANYTVGNTWYRGYDPFTPAADQIGQPWMIQRAWDSIDPIFSVNDKALACNTPATAPTSYIPIRAGENITAVYWYWLHPVGPMTAWLARCDGDCRDADVNEARWFKIWEAGLLSGPNLAEGMWYQKAFQNWDGSPDLWPVTIPAGLKSGLYMIRHEILSIHVEDKPQFYPECAHLNVTGGGDLLPPDEFLVKFPGAYKEDNPSIKINIYSDQYANTTNYTIPGGPIWDG

SEQ ID NO: 27 - protein, 478 aa, Thielavia terrestris
MMPSLVRFSMGLATAFASLSTAHTVFTTLFINGVDQGDGTCIRMAKKGSVCTHPIAGGLDSPDMACGRDGQQAVAFTCPAPAGSKLSFEFRMWADASQPGSIDPSHLGSTAIYLKQVSNISSDSAAGPGWFKIYAEGYDTAAKKWATEKLIDNGGLLSIELPPTLPAGYYLARSEIVTIQNVTNDHVDPQFYVGCAQLFVQGPPTTPTVPPDRLVSIPGHVHASDPGLTFNIWRDDPSKTAYTVVGPAPFSPTAAPTPTSTNTNGQQQQQQQQAIKQTDGVIPADCQLKNANWCGAEVPAYADEAGCWASSADCFAQLDACYTSAPPTGSRGCRLWEDWCTGIQQGCRAGRWRGPPPFHGEGAAAETASAGRGGARIAAVAGCGGGTGDMVEEVFLFYWDACSGWRRSRGGGSILARLILHVLLPLLRPRRAPRVHLLLFHLYLNFCYPGTSGFYNRLSIKLGIWPSKMSPDVAHYVK

SEQ ID NO: 28 - protein, 230 aa, Thielavia terrestris
MQLLVGLLLAAVAARAHYTFPRLVVNGQPEDKDWSVTRMTKNAQSKQGVQDPTSPDIRCYTSQTAPNVATVPAGATVHYISTQQINHPGPTQYYLAKVPAGSSAKTWDGSGAVWFKISTTMPYLDNNKQLVWPNQNTYTTVNTTIPADTPSGEYLLRVEQIALHLASQPNGAQFYLACSQIQITGGGNGTPGPLVALPGAYKSNDPGILVNIYSMQPGDYKPPGPPVWSG

SEQ ID NO: 29 - protein, 257 aa, Thielavia terrestris
MKLYLAAFLGAVATPGAFAHQIHGILLVNGTETPEWKYVRDVAWEGAYEPEKYPNTEFFKTPPQTDINNPNITCGRNAFDSASKTETADILAGSEVGFRVSWDGNGKYGVFWHPGPGQIYLSRAPNDDLEDYRGDGDWFKIATGAAVSNTEWLLWNKHDFNFTIPKTTPPGKYLMRIEQFMPSTVEYSQWYVNCAHVNIIGPGGGTPTGFARFPGTYTVDDPGIKVPLNQIVNSGELPQDQLRLLEYKPPGPALWTG

SEQ ID NO: 30 - protein, 251 aa, Thermoascus crustaceus
MAFSQIMAITGVFLASASLVAGHGFVQNIVIDGKSYGGYIVNQYPYMSDPPEVVGWSTTATDLGFVDGTGYQGPDIICHRGAKPAALTAQVAAGGTVKLEWTPWPDSHHGPVINYLAPCNGDCSTVDKTQLKFFKIAQAGLIDDNSPPGIWASDNLIAANNSWTVTIPTTTAPGNYVLRHEIIALHSAGNKDGAQNYPQCINLKVTGNGSGNPPAGALGTALYKDTDPGILINIYQKLSSYVIPGPALYTG

SEQ ID NO: 31 - protein, 349 aa, Thermoascus crustaceus
MSFSKILAIAGAITYASSAAAHGYVQGIVVDGSYYGGYMVTQYPYTAQPPELIAWSTKATDLGFVDGSGYTSPDIICHKGAEPGAQSAKVAAGGTVELQWTAWPESHKGPVIDYLAACDGDCSSVDKTALKFFKIDESGLIDGNGAGTWASDTLIKNNNSWTVTIPSTIASGNYVLRHEIIALHSAGNKDGAQNYPQCINLEVTGSGTENPAGTLGTALYTDTDPGLLVNIYQGLSNYSIPGPALYSGNSDNAGSLNPTTTPSIQNAAAAPSTSTASVVTDSSSATQTASVAATTPASTSAVTASPAPDTGSDVTKYLDSMSSDEVLTLVRGTLSWLVSNKKHARDLSH

SEQ ID NO: 32 - protein, 436 aa, Thermoascus crustaceus
MLSFIPTKSAALTTLLLLGTAHAHTLMTTMFVDGVNQGDGVCIRMNNDGGTANTYIQPITSKDIACGIQGEIGASRVCPVKASSTLTFQFREQPNNPNSSPLDPSHKGPAAVYLKKVDSAIASNNAAGDSWFKIWESVYDESTGKWGTTKMIENNGHISVKVPDDIEGGYYLARTELLALHSADQGDPQFYVGCAQLFIDSDGTAKPPTVSIGEGTYDLSMPAMTYNIWETPLALPYPMYGPPVYTPGSGSGSVRATSSSAVPTATESSFVEERANPVTANSVYSARGKFKTWIDKLSWRGKVRENVRQAAGRRSTLVQTVGLKPKGCIFVNGNWCGFEVPDYNDAESCWAASDNCWKQSDACWNKTQPTGYNNCQIWQDKKCKVIQDSCSGPNPHGPPNKGKDLTPEWPPLKGSMDTFSKRTIGYRDWIVRRRGA

SEQ ID NO: 33 - protein, 320 aa, Talaromyces stipitatus
MPSTKVAALSAVLALASTVAGHGFVQNIVIDGKSYTGYLVNQYPYQSNPPAVIGWSTTATDLGFVDGSEYTNPDIICHKNAKPGQLSAPVAAGGKVELEWTTWPESHHGPVISYLANCNGDCTTVDKTKLEFVKIDQRGLIDDSNPPGTWAADQLIAANNSWTVTIPESIAPGNYVLRHEIIALHSANNANGAQNYPQCINLQITGSGTANPSGTPGEKLYTPTDPGILVNIYQSLSTYAIPGPTLWSGAAAHVVATATGSATGVASATATPTTLVTAVSSRTGAPSVVTPEAPSVTSFAPVVTVTDVVTVTTVITTTIS

(19) United States
(12) Patent Application Publication
(10) Pub. No.: US 2013/0302879 A1
(43) Pub. Date: Nov. 14, 2013

(54) Processes for Treating Textile with Polypeptide Having Cellulolytic Enzyme Enhancing Activity

(71) Applicants: Weijan Lai, Beijing (CN); Guifang Wu, Beijing (CN)

(72) Inventors: Weijan Lai, Beijing (CN); Guifang Wu, Beijing (CN)

(73) Assignee: Novozymes A/S, Bagsvaerd (DK)

(21) Appl. No.: 13/997,772

(22) PCT Filed: Dec. 14, 2011
(86) PCT No.: PCT/CN2011/083996

§ 371 Date: Jul. 25, 2013

(30) Foreign Application Priority Data
Dec. 30, 2010 (CN) PCT/CN2010/080530

(51) Int. Cl. D06M 16/00; C12N 9/42

(52) U.S. Cl. CPC D06M 16/003; C12N 9/2437
USPC 435/192; 435/263; 435/209; 435/198; 435/201; 435/189; 435/193; 8/401

(57) ABSTRACT

The present invention relates to the use of glycosyl hydrolase family (61) polypeptides in the presence of cellulases for textile manufacture as well as a textile composition comprising glycosyl hydrolase family (61) polypeptides and cellulases.

### REFERENCE TO SEQUENCE LISTING

This application contains a Sequence Listing in computer readable form. The computer readable form is incorporated herein by reference.

### FIELD OF THE INVENTION

The present invention relates to the use of glycosyl hydrolase family 61 polypeptides as enhancers of cellulases in textile manufacture as well as a textile composition comprising glycosyl hydrolase family 61 polypeptides and cellulases.

### BACKGROUND OF THE INVENTION

There is a wide spectrum of industrial applications of cellulases. In the textile industry, cellulases are used in denim finishing to create a fashionable stone washed appearance in denim cloths in a biostoning process. Cellulases are also used, for instance, to clean fuzz and prevent formation of pills on the surface of cotton garments.

A general problem associated with enzymatic stone washing is the backstaining caused by redeposition of removed Indigo dye during or after abrasion. The “backstaining” or “redeposition” of Indigo dye reduces the desired contrast between the white and indigo dyed yarns and it can be most easily noted on the reverse side of denim and the interior pockets (as increased blueness). On the face side this may be seen as reduced contrast between dyed areas and areas from which dye has been removed during biostoning. In order to remove the dye, the denim manufacturers are using large amounts of surfactants to make parts white again in a soaping process. The heavy washing condition causes colour change or colour-fading problems for finished denim products. Also additional water has to be used in the subsequent soaping process. The problem of redeposition or backstaining of dye during stonewashing has also been addressed by adding anti-redeposition chemicals, such as surfactants or other agents into the cellulase wash.

Also the use of different cellulases with less specific activity on denim has been tried. WO9407983 describes the use of a cellulase to inhibit the backstaining of denim. WO9429426 and WO9325655 describe backstaining inhibition by treatment with a redoposition cellulase composition and added protease as an improvement over the use of redeposition cellulase alone. WO9709410 describes that the addition of a certain type of cellulase to another cellulase having abrading activity reduces biostoning. The additional cellulase belongs to family 5 or 7, but it has no significant abrading effect by itself. WO0192453 discloses backstaining reduction by treating textile with a cutinase.

However, there is still a need for improved benefit of enzymatic textile treatment, including enhancing the efficiency of the enzymes to their substrates. In particular, there is a continuous need for more efficient enzyme composition to improve the economics of the process. The present invention aims to meet these needs.

### SUMMARY OF THE INVENTION

The present invention relates to a method for treating textile with a glycosyl hydrolase family 61 (GH61) polypeptide in the presence of a cellulase in an aqueous solution.

The present invention also relates to a textile composition comprising a glycosyl hydrolase family 61 polypeptide and a cellulase.

In an embodiment, the method can be applied in a biostoning process to form localized variation of color density in the surface of a dyed cellulosic or cellulose-containing fabric, by contacting dyed cellulosic or cellulose-containing fabric with a glycosyl hydrolase family 61 polypeptide and a cellulase.

In one embodiment, the process of the invention is applied to any type of dyed cellulosic fabric where it is desired to form localized variation of color density in the surface. An example of particular commercial interest is denim, particularly indigo-dyed denim for use in blue jeans, etc.

In one embodiment, a number of enzymes can be used together with cellulase and GH61 during biostoning process, which comprises one or more enzymes selected from the group consisting of proteases, lipases, cutinases, amylases, pectinases, hemicellulases, oxidoreductases, peroxidases, laccases, and transferases.

In an embodiment, the method can be applied in a biopolishing process to reduce pilling formation, by contacting cellulosic or cellulose-containing fabric with a glycosyl hydrolase family 61 polypeptide in the presence of a cellulase in an aqueous solution.

In one embodiment, the method and composition may further comprise a cosubstance, such as cysteine.

In some embodiments, the method for manufacturing textile is provided. In some embodiments, the textile is manufactured from fabric to garment.

In some embodiments, the cellulase used in the present invention is cellulase having abrasion effect. In some embodiment, the cellulase is endoglucanase.

In the present invention, GH61 polypeptides can enhance the efficiency of the cellulase to its substrate with at least one of the following benefits: increased denim abrasion level, low backstaining level, promoting the dye release from the textile, colour clarification and reduction of pilling formation.

GH61 polypeptides have previously been applied in baking, where they have been shown to have an anti-staling effect, WO 04/031378. Furthermore, GH61 polypeptides have been applied in the conversion of cellulosic feedstock into ethanol, WO 05/074647, WO 05/074656, WO 07/089,290, and WO 09/033,071. There is, however, no indication in these applications that GH61 polypeptides are capable of enhancing the effect in textile manufacturing process.

### DETAILED DESCRIPTION OF THE INVENTION

The invention will now be described in detail by way of reference using the following definitions and examples. All patents and publications, including all sequences disclosed within such patents and publications, referred to herein are expressly incorporated by reference.

As used herein, the singular terms “a”, “an,” and “the” include the plural reference unless the context clearly indicates otherwise.

### DEFINITIONS

### Glycoside Hydrolase Family 61 (GH61) Polypeptides

The term “glycoside hydrolase family 61” or “GH61” is defined herein as a polypeptide falling into the glycoside hydrolase family 61 according to Henrissat B., 1991, Biochem. J. 280: 309-316, and Henrissat B., and Bairoch A., 1996, Biochem. J. 316: 695-696.

The present invention relates to the use of isolated GH61 polypeptides in general. A GH61 polypeptide useful in the present invention may be obtained from microorganisms of any genus. For purposes of the present invention, the term “obtained from” as used herein in connection with a given source shall mean that the polypeptide encoded by a nucleotide sequence is produced by the source in which it is naturally present or by a strain in which the nucleotide sequence from the source has been inserted. In a preferred aspect, the polypeptide obtained from a given source is secreted extracellularly.

A polypeptide of the present invention may be a bacterial polypeptide. For example, the polypeptide may be a gram positive bacterial polypeptide such as a Bacillus polypeptide, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis polypeptide; or a Streptomyces polypeptide, e.g., a Streptomyces lividans or Streptomyces murinus polypeptide; or a gram negative bacterial polypeptide, e.g., an E. coli or a Pseudomonas sp. polypeptide.

A polypeptide of the present invention may also be a fungal polypeptide, and more preferably a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Chaetomium, Cryptococcus, Filibasidium, Fusarium, Humicola, Magnaporthe, Mucor, Myceliophthora, Neocallimastix, Neurospora, Paecilomyces, Penicillium, Piromyces, Poronia, Schizophyllum, Talaromyces, Thermoascus, Thielevia, Tolypocladium, Trichoderma or Verticillium polypeptide.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme detergency enhancing effect.

In another preferred aspect, the polypeptide is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Aspergillus terreus, Chaetomium globosum, Coprinus cinereus, Diplodia gossyppina, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Magnaporthe grisea, Mucor miehei, Myceliophthora thermophila, Neurospora crassa, Penicillium purpurogenum, Phanerochaete chlysosporium, Poronia punctata, Pseudoplectania nigrella, Thermoascus aurantiacus, Thielavia terrestris, Trichoderma harzianum, Trichoderma koningii, Trichoderma longibrachiatum, Trichoderma reesei, Trichoderma viride, Trichophaea saccata, Verticillium tenerum or Talaromyces stipitatus polypeptide.

In the processes of the present invention, any GH61 polypeptide having cellulolytic enhancing activity can be used.

For purposes of the present invention, cellulolytic enhancing activity is determined by measuring the increase in the abrasion level under condition as specified in Example 1, by treatment of cellulolytic enzyme in Launder-O-Meter (LOM) at 55° C. and pH 6.5 for 2 hours, with cellulase dosage of 0.05 mg/g fabric and GH61 dosage of 0.042 mg/g fabric. In a preferred embodiment of the present invention, the abrasion level is increased by at least 0.08 Delta L* unit, preferably at least 0.1, more preferably at least 0.2, more preferably at least 0.4, more preferably at least 0.5, more preferably at least 0.6, more preferably at least 0.7, more preferably at least 0.8, more preferably at least 0.9, even more preferably at least 1, even more preferably at least 1.2, and most preferably at least 1.4 Delta L* unit when the cellulase (or cellulolytic enzyme) is combined with a glycosyl hydrolase family 61 polypeptide as compared to the result when the cellulase is used without the glycosyl hydrolase family.

In a first aspect, the GH 61 polypeptide having cellulolytic enhancing activity comprises the following motifs:
[ILMV]-P—X(4,5)-G-X—Y-[ILMV]-X—R—X-[EQ]-X(4)-[HNQ] and [FW]-[TF]-K-[AIV],
wherein X is any amino acid, X(4,5) is any amino acid at 4 or 5 contiguous positions, and X(4) is any amino acid at 4 contiguous positions.

The polypeptide comprising the above-noted motifs may further comprise:
H—X(1,2)-G-P—X(3)-[YW]-[AILMV],
[EQ]X—Y—X(2)-C—X-[EHQN]-[FILV]-X-[ILV], or
H—X(1,2)-G-P—X(3)-[YW]-[AILMV] and [EQ]-X—Y—X(2)-C—X-[EHQN]-[FILV]-X-[ILV],
wherein X is any amino acid, X(1,2) is any amino acid at 1 position or 2 contiguous positions, X(3) is any amino acid at 3 contiguous positions, and X(2) is any amino acid at 2 contiguous positions. In the above motifs, the accepted IUPAC single letter amino acid abbreviation is employed.

In a preferred aspect, the polypeptide having cellulolytic enhancing activity further comprises H—X(1,2)-G-P—X(3)-[YW]-[AILMV]. In another preferred aspect, the isolated polypeptide having cellulolytic enhancing activity further comprises [EQ]-X—Y—X(2)-C—X-[EHQN]-[FILV]-X-[ILV]. In another preferred aspect, the polypeptide having cellulolytic enhancing activity further comprises H—X(1,2)-G-P—X(3)-[YW]-[AILMV] and [EQ]-X—Y—X(2)-C—X-[EHQN]-[FILV]-X-[ILV].

In a second aspect, the polypeptide having cellulolytic enhancing activity comprises the following motif:
[ILMV]-P-x(4,5)-G-x-Y-[ILMV]-x-R-x-[EQ]-x(3)-A-[HNQ],
wherein x is any amino acid, x(4,5) is any amino acid at 4 or 5 contiguous positions, and x(3) is any amino acid at 3 contiguous positions. In the above motif, the accepted IUPAC single letter amino acid abbreviation is employed.

In a third aspect, the polypeptide having cellulolytic enhancing activity comprises an amino acid sequence that has a degree of identity to the mature polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33 of at least 60%, e.g., at least 65%, at least 70%, at least 75%, at least 80%, at least 85%, at least 90%, at least 91%, at least 92%, at least 93%, at least 94%, or at least 95%, at least 96%, at least 97%, at least 98%, at least 99%, or at least 100%.

In a sixth aspect, the polypeptide having cellulolytic enhancing activity is an artificial variant comprising a substitution, deletion, and/or insertion of one or more (or several) amino acids of the mature polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, or SEQ ID NO: 33; or a homologous sequence thereof.

More preferably, the GH61 polypeptide is a variant with a substitution, deletion, and/or insertion of at least 10, 9, 8, 7, 6, 5, 4, 3, 2 or 1 amino acids of any one of the mature polypeptides of SEQ ID NO: 1 to 32.

The parameter “identity” as used herein describes the relatedness between two amino acid sequences or between two nucleotide sequences. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; http://emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Substantially homologous polypeptides of the sequences described above are characterized as having one or more (several) amino acid a substitutions, deletions, and/or insertions in the mature polypeptide. Preferably, amino acid changes are of a minor nature, that is conservative amino acid substitutions or insertions that do not significantly affect the folding and/or activity of the protein; small deletions, typically of one to about 9 amino acids, preferably from one to about 15 amino acids and most preferably from one to about 30 amino acids; small amino- or carboxyl-terminal extensions, such as an amino-terminal methionine residue; a small linker peptide of up to about five to ten residues, preferably from 10 to 15 residues and most preferably from 20 to 25 residues, or a small extension that facilitates purification by changing net charge or another function, such as a poly-histidine tag, an antigenic epitope, protein A, a CBM or a another binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., enzyme detergency enhancing effects) to identify amino acid residues that are critical to the activity of the molecule. See also, Hilton et al., 1996, J. Biol. Chem. 271: 4699-4708. Three dimensional structures, such as alpha-helixes, beta-sheets, as well as metal binding site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. Especially, Karkehabadi et al., 2008 J. Mol. Biol. 383: 144-154 describes the crystal structure of GH61 from Hypocrea jecorina. The identities of essential amino acids can also be inferred from analysis of identities with polypeptides that are related to a polypeptide according to the invention.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

### Cosubstance

The addition of a cosubstance together with GH61 polypeptides can enhance the enzymatic efficiency even further with at least one of the following benefits: increased abrasion effect, low backstaining level, and reduced pilling formation etc.

In one aspect, the GH61 polypeptide having cellulolytic enhancing activity is used in the presence of a soluble activating divalent metal cation according to WO 2008/151043, e.g., manganese sulfate.

In one aspect, the GH61 polypeptide having cellulolytic enhancing activity is used in the presence of a dioxy compound, a bicylic compound, a heterocyclic compound, a nitrogen-containing compound, or a sulfur-containing compound.

The dioxy compound may include any suitable compound containing two or more oxygen atoms. In some aspects, the dioxy compounds contain a substituted aryl moiety as described herein. The dioxy compounds may comprise one or more (several) hydroxyl and/or hydroxyl derivatives, but also include substituted aryl moieties lacking hydroxyl and hydroxyl derivatives. Non-limiting examples of dioxy compounds include pyrocatechol or catechol; caffeic acid; 3,4-dihydroxybenzoic acid; 4-tert-butyl-5-methoxy-1,2-benzenediol; pyrogallol; gallic acid; methyl-3,4,5-trihydroxybenzoate; 2,3,4-trihydroxybenzophenone; 2,6-dimethoxyphenol; sinapinic acid; 3,5-dihydroxybenzoic acid; 4-chloro-1,2-benzenediol; 4-nitro-1,2-benzenediol; tannic acid; ethyl gallate; methyl glycolate; dihydroxyfumaric acid; 2-butyne-1,4-diol; (croconic acid; 1,3-propanediol; tartaric acid; 2,4-pentanediol; 3-ethyoxy-1,2-propanediol; 2,4,4′-trihydroxybenzophenone; cis-2-butene-1,4-diol; 3,4-dihydroxy-3-cyclobutene-1,2-dione; dihydroxyacetone; acrolein acetal; methyl-4-hydroxybenzoate; 4-hydroxybenzoic acid; and methyl-3,5-dimethoxy-4-hydroxybenzoate; or a salt or solvate thereof.

The bicyclic compound may include any suitable substituted fused ring system as described herein. The compounds may comprise one or more (several) additional rings, and are not limited to a specific number of rings unless otherwise stated. In one aspect, the bicyclic compound is a flavonoid. In another aspect, the bicyclic compound is an optionally substituted isoflavonoid. In another aspect, the bicyclic compound is an optionally substituted flavylium ion, such as an optionally substituted anthocyanidin or optionally substituted anthocyanin, or derivative thereof. Non-limiting examples of bicyclic compounds include epicatechin; quercetin; myricetin; taxifolin; kaempferol; morin; acacetin; naringenin; isorhamnetin; apigenin; cyanidin; cyanin; kuromanin; keracyanin; or a salt or solvate thereof.

The heterocyclic compound may be any suitable compound, such as an optionally substituted aromatic or non-aromatic ring comprising a heteroatom, as described herein. In one aspect, the heterocyclic is a compound comprising an optionally substituted heterocycloalkyl moiety or an optionally substituted heteroaryl moiety. In another aspect, the optionally substituted heterocycloalkyl moiety or optionally substituted heteroaryl moiety is an optionally substituted 5-membered heterocycloalkyl or an optionally substituted 5-membered heteroaryl moiety. In another aspect, the optionally substituted heterocycloalkyl or optionally substituted heteroaryl moiety is an optionally substituted moiety selected from pyrazolyl, furanyl, imidazolyl, isoxazolyl, oxadiazolyl, oxazolyl, pyrrolyl, pyridyl, pyrimidyl, pyridazinyl, thiazolyl, triazolyl, thienyl, dihydrothieno-pyrazolyl, thianaphthenyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, benzoisazolyl, dimethylhydantoin, pyrazinyl, tetrahydrofuranyl, pyrrolinyl, pyrrolidinyl, morpholinyl, indolyl, diazepinyl, azepinyl, thiepinyl, piperidinyl, and oxepinyl. In another aspect, the optionally substituted heterocycloalkyl moiety or optionally substituted heteroaryl moiety is an optionally substituted furanyl. Non-limiting examples of heterocyclic compounds include (1,2-dihydroxyethyl)-3,4-dihydroxyfuran-2(5H)-one; 4-hydroxy-5-methyl-3-furanone; 5-hydroxy-2(5H)-furanone; [1,2-dihydroxyethyl]furan-2,3,4(5H)-trione; α-hydroxy-γ-butyrolactone; ribonic γ-lactone; aldohexuronicaldohexuronic acid γ-lactone; gluconic acid δ-lactone; 4-hydroxycoumarin; dihydrobenzofuran; 5-(hydroxymethyl)furfural; furoin; 2(5H)furanone; 5,6-dihydro-2H-pyran-2-one; and 5,6-dihydro-4-hydroxy-6-methyl-2H-pyran-2-one; or a salt or solvate thereof.

The nitrogen-containing compound may be any suitable compound with one or more nitrogen atoms. In one aspect, the nitrogen-containing compound comprises an amine, imine, hydroxylamine, or nitroxide moiety. Non-limiting examples of nitrogen-containing compounds include acetone oxime; violuric acid; pyridine-2-aldoxime; 2-aminophenol; 1,2-benzenediamine; 2,2,6,6-tetramethyl-1-piperidinyloxy; 5,6,7,8-tetrahydrobiopterin; 6,7-dimethyl-5,6,7,8-tetrahydropterine; and maleamic acid; or a salt or solvate thereof.

The quinone compound may be any suitable compound comprising a quinone moiety as described herein. Non-limiting examples of quinone compounds include 1,4-benzoquinone; 1,4-naphthoquinone; 2-hydroxy-1,4-naphthoquinone; 2,3-dimethoxy-5-methyl-1,4-benzoquinone or coenzyme Q₀; 2,3,5,6-tetramethyl-1,4-benzoquinone or duroquinone; 1,4-dihydroxyanthraquinone; 3-hydroxy-1-methyl-5,6-indolinedione or adrenochrome; 4-tert-butyl-5-methoxy-1,2-benzoquinone; pyrroloquinoline quinone; or a salt or solvate thereof.

The sulfur-containing compound may be any suitable compound comprising one or more sulfur atoms. In one aspect, the sulfur-containing comprises a moiety selected from thionyl, thioether, sulfinyl, sulfonyl, sulfamide, sulfonamide, sulfonic acid, and sulfonic ester. Non-limiting examples of sulfur-containing compounds include ethanethiol; 2-propanethiol; 2-propene-1-thiol; 2-mercaptoethanesulfonic acid; benzenethiol; benzene-1,2-dithiol; cysteine; methionine; glutathione; cystine; or a salt or solvate thereof.

In one aspect, the amount of such a compound described above to cellulosic material as a molar ratio to glucosyl units of cellulose is about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁴ to about 10⁻¹, about 10⁻³ to about 10⁻¹, and about 10⁻³ to about 10⁻².

In another aspect, an effective amount of such a compound described above is about 0.1 μM (micromolar) to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, and about 0.1 mM to about 1 mM.

The term “liquor” means the solution phase, either aqueous, organic, or a combination thereof.

In one aspect, an effective amount of the liquor to cellulose is about 10⁻⁶ to about 10 g per g of cellulose, e.g., about 10⁻⁶ to about 7.5 g, about 10⁻⁶ to about 5, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, and about 10⁻³ to about 10⁻² g per g of cellulose.

### Textile

The term “textiles” used herein is meant to include fibers, yarns, fabrics and garments.

Fabric can be constructed from fibers by weaving, knitting or non-woven operations. Weaving and knitting require yarn as the input whereas the non-woven fabric is the result of random bonding of fibers (paper can be thought of as non-woven). In the present context, the term “fabric” is also intended to include fibers and other types of processed fabrics.

According to the invention, the method of the invention may be applied to any textile known in the art (woven, knitted, or non-woven). In particular the process of the present invention may be applied to cellulose-containing or cellulosic textile, such as cotton, viscose, rayon, ramie, linen, lyocell (e.g., Tencel, produced by Courtaulds Fibers), or mixtures thereof, or mixtures of any of these fibers together with synthetic fibres (e.g., polyester, polyamid, nylon) or other natural fibers such as wool and silk., such as viscose/cotton blends, lyocell/cotton blends, viscose/wool blends, lyocell/wool blends, cotton/wool blends; flax (linen), ramie and other fabrics based on cellulose fibers, including all blends of cellulosic fibers with other fibers such as wool, polyamide, acrylic and polyester fibers, e.g., viscose/cotton/polyester blends, wool/cotton/polyester blends, flax/cotton blends etc.

### Textile Manufacturing Process

The processing of a fabric, such as of a cellulosic material, into material ready for garment manufacture involves several steps: spinning of the fiber into a yarn; construction of woven or knit fabric from the yarn; and subsequent preparation processes, dyeing/printing and finishing operations. Preparation processes are necessary for removing natural and man-induced impurities from fibers and for improving their aesthetic appearance and processability prior to for instance dyeing/printing and finishing. Common preparation processes comprise desizing (for woven goods), scouring, and bleaching, which produce a fabric suitable for dyeing or finishing.

Woven fabric is constructed by weaving “filling” or “weft” yarns between warp yarns stretched in the longitudinal direction on the loom. The warp yarns must be sized before weaving in order to lubricate and protect them from abrasion at the high speed insertion of the filling yarns during weaving. Common size agents are starches (or starch derivatives and modified starches), poly(vinyl alcohol), carboxylmethyl cellulose (i.e. CMC) where starches are dominant. Paraffin, acrylic binders and variety of lubricants are often included in the size mix. The filling yarn can be woven through the warp yarns in a “over one—under the next” fashion (plain weave) or by “over one—under two” (twill) or any other myriad of permutations. Generally, dresses, shirts, pants, sheeting's, towels, draperies, etc. are produced from woven fabric. After the fabric is made, size on the fabric must be removed again (i.e. desizing).

Knitting is forming a fabric by joining together interlocking loops of yarn. As opposed to weaving, which is constructed from two types of yarn and has many “ends”, knitted fabric is produced from a single continuous strand of yarn. As with weaving, there are many different ways to loop yarn together and the final fabric properties are dependent both upon the yarn and the type of knit. Underwear, sweaters, socks, sport shirts, sweat shirts, etc. are derived from knit fabrics.

### Desizinq

Desizing is the degradation and/or removal of sizing compounds from warp yarns in a woven fabric. Starch is usually removed by an enzymatic desizing procedure. In addition, oxidative desizing and chemical desizing with acids or bases are sometimes used.

In some embodiments, the desizing enzyme is an amylolytic enzyme, such as an alpha-amylase, a beta-amylase, a mannanases, a glucoamylases, or a combination thereof.

Suitable alpha and beta-amylases include those of bacterial or fungal origin, as well as chemically or genetically modified mutants and variants of such amylases. Suitable alpha-amylases include alpha-amylases obtainable from Bacillus species. Suitable commercial amylases include but are not limited to OPTISIZE® NEXT, OPTISIZE® FLEX and OPTISIZE® COOL (all from Genencor International Inc.), and DURAMYL™, ERMAMYL™, FUNGAMYL™ TERMAMYL™, AUQAZYME™ and BAN™ (all available from Novozymes A/S, Bagsvaerd, Denmark).

Other suitable amylolytic enzymes include the CGTases (cyclodextrin glucanotransferases, EC 2.4.1.19), e.g., those obtained from species of Bacillus, Thermoanaerobactor or Thermoanaero-bacterium.

### Scouring

Scouring is used to remove impurities from the fibers, to swell the fibers and to remove seed coat. It is one of the most critical steps. The main purposes of scouring is to a) uniformly clean the fabric, b) soften the motes and other trashes, c) improve fabric absorbency, d) saponify and solubilize fats, oils, and waxes, and e) minimize immature cotton. Sodium hydroxide scouring at about boiling temperature is the accepted treatment for 100% cotton, while calcium hydroxide and sodium carbonate are less frequently used. Synthetic fibers are scoured at much milder conditions. Surfactant and chelating agents are essential for alkaline scouring. Enzymatic scouring has been introduced, wherein cellulase, hemicellulase, pectinase, lipase, and protease are all reported to have scouring effects.

### Bleaching

Bleaching is the destruction of pigmented color and/or colored impurities as well as seed coat fragment removal. It is the most critical chemical treatment since a balance between the degrees of whiteness with fiber damage must be maintained. Bleaching is performed by the use of oxidizing or reducing chemistry. Oxidizing agents can be further subdivided into those that employ or generate: a) hypochlorite (OCl⁻), b) chloride dioxide (ClO₂), and hydroperoxide species (OOH⁻and/or OOH). Reducing agents are typical sulfur dioxide, hydrosulfite salts, etc. Enzymatic bleaching using glucose oxidase has been reported. Traditionally, hydrogen peroxide is used in this process.

### Printing and Dyeing

Printing or dyeing of textiles is carried out by applying dyes to the textile by any appropriate method for binding the dyestuff to the fibres in the textiles. The dyeing of textiles is for example carried out by passing the fabric through a concentrated solution of dye, followed by storage of the wet fabric in a vapour tight enclosure to permit time for diffusion and reaction of the dye with the fabric substrate prior to rinsing off un-reacted dye. Alternatively, the dye may be fixed by subsequent steaming of the textile prior to rinsing. The dyes include synthetic and natural dyes. Typical dyes are those with anionic functional groups (e.g. acid dyes, direct dyes, Mordant dyes and reactive dyes), those with cationic groups (e.g. basic dyes), those requiring chemical reaction before application (e.g. vat dyes, sulphur dyes and azoic dyes), disperse dyes and solvent dyes.

Excess soluble dyestuff not bound to the fibres must be removed after dyeing to ensure fastness of the dyed textiles and to prevent unwanted dye transfer during laundering of the textiles by the consumer. Generally, a large amount of water is required for complete removal of excess dye. In a conventional process, the printed or dyed textile is first rinsed with cold water, then washed at high temperature with the addition of a suitable additive to decrease backstaining, like poly(vinylpyrrolidone) (PVP).

An enzymatic process for removal of excess dye from dyed fabric with a rinse liquor comprising at least one peroxidise, an oxidase agent and at least one mediator, such as liquor comprising a peroxidase, hydrogen peroxidise and a mediator like 1-hydroxy-benzotriazole is disclosed in WO99/34054.

### Biopolishing

As used herein, the term “biopolishing”, “depilling” and “anti-pilling” are interchangeable.

Most cotton fabrics and cotton blend fabrics have a handle appearance that is rather hard and stiff without the application of finishing components. The fabric surface also is not smooth because small fuzzy microfibrils protrude from it. In addition, after a relatively short period of wear, pilling appears on the fabric surface thereby giving it an unappealing, worn look.

Biopolishing is a method to treat cellulosic fabrics during their manufacturing by enzymes such as cellulases, which improves fabric quality with respect to “reduced pilling formation”. The most important effects of biopolishing can be characterised by less fuzz and pilling, increased gloss/luster, improved fabric handle, increased durable softness and/or improved water absorbency. Biopolishing usually takes place in the wet processing of the manufacture of knitted and woven fabrics or garments. Wet processing comprises such steps as e.g., desizing, scouring, bleaching, washing, dying/printing and finishing. Biopolishing could be performed as a separate step after any of the wetting steps or in combination with any of those wetting steps.

The method of the present invention of treating textile with a GH61 polypeptide in the presence of cellulase in an aqueous solution can be applied to a biopolishing process.

In one embodiment, the invention provides a method for obtaining a cellulosic or cellulose-containing textile having a reduced tendency to pilling formation, the method comprising treating textile with a GH61 polypetide in the presence of cellulase in an aqueous solution. In this embodiment, the method of biopolishing can be applied to yarn, fabric or garment.

In the present context, the term “reduced pilling formation” is intended to mean a resistance to the formation of pills on the surface of the treated (biopolished) fabric surface according to the method of the present invention, in comparison with fabric without enzymatic treatment. For the purpose of the present invention, the pilling formation may be tested according the description of “pilling notes test” in the material and method section. The results of the test is expressed in terms of “pilling notes” which is a rating on a scale from pilling note 1 (heavy pill formation) to pilling note 5 (no pill formation), allowing ¼ pilling notes.

Since the enzymes of the present invention catalyze hydrolysis of the cellulosic fibre surface, the enzymatic action will eventually result in a weight loss of fibre or fabric. In a preferred embodiment, even though the biopolishing is carried out in such a way so as to obtain a controlled, partial hydrolysis of the fibre surface, a proper polishing effect without excessive loss of fabric strength has hitherto been obtained.

It is to be understood that the method of the invention can be carried out in any conventional wet textile processing step, preferably after the desizing or bleaching of the textile fabric, either simultanously with a conventional (well-known) process step or as an additional process step. The method will typically be accomplished in high-speed circular systems such as jet-overflow dyeing machines, high-speed winches and jiggers. An example of a useful Highspeed system is the “Aero 1000” manufactured by Biancalani, Italy. The method of the present invention can be carried out in a batch, continuous or semi-continuous apparatus, such as a JBox, on a Pad-Roll or in a Pad-Bath.

### Manufacturing of Denim Fabric

Some dyed fabric such as denim fabric, requires that the yarns are dyed before weaving. For denim fabric, the warp yarns are dyed for example with indigo, and sized before weaving. Preferably the dyeing of the denim yarn is a ring-dyeing. A preferred embodiment of the invention is ring-dyeing of the yarn with a vat dye such as indigo, or an indigo-related dye such as thioindigo, or a sulfur dye, or a direct dye, or a reactive dye, or a naphthol. The yarn may also be dyed with more than one dye, e.g., first with a sulphur dye and then with a vat dye, or vice versa.

Preferably, the yarns undergo scouring and/or bleaching before they are dyed, in order to achieve higher quality of denim fabric. In general, after woven into dyed fabric, such as denim, the dyed fabric or garment proceeds to a desizing stage, preferably followed by a biostoning step and/or a color modification step.

The desizing process as used herein is the same process as mentioned above in the context.

After desizing, the dyed fabric undergoes a biostoning step. The biostoning step can be performed with enzymes or pumice stones or both. As used herein, the term “biostoning”, “stone washing” and “abrasion” are interchangeable, which means agitating the denim in an aqueous medium containing a mechanical abrasion agent such as pumice, an abrading cellulase or a combination of these, to provide a “stone-washed” look (i.e. a localized variation of colour density in the denim surface). In all cases, mechanical action is needed to remove the dye, and the treatment is usually carried out in washing machines, like drum washers, belly washers. As a result of uneven dye removal there are contrasts between dyed areas and areas from which dye has been removed, this appears as a localized variation of colour density. Treatment with cellulase can completely replace treatment with pumice stones. However, cellulase treatment can also be combined with pumice stone treatment, when it is desired to produce a heavily abraded finish.

For the purpose of the present invention, abrasion level is used to indicate the localized variation of colour density, which is measured under condition as specified in Example 1. The effect of cellulolytic enhancing activity of GH61 is determined by measuring the increase in the abrasion level under conditions as specified in Example 1, by treatment of cellulolytic enzyme in LOM at 55° C. and pH 6.5 for 2 hours, with cellulase dosage of 0.05 mg/g fabric and GH61 dosage of 0.042 mg/g fabric. In a preferred embodiment of the present invention, the abrasion level is increased by at least 0.08 Delta L* unit, preferably at least 0.1, more preferably at least 0.2, more preferably at least 0.4, more preferably at least 0.5, more preferably at least 0.6, more preferably at least 0.7, more preferably at least 0.8, more preferably at least 0.9, even more preferably at least 1, even more preferably at least 1.2, and most preferably at least 1.4 Delta L* unit as compared to the result when the cellulase is used without GH61.

The dyestuff removed from the denim material after the treatment with cellulase or by a conventional washing process may cause “backstaining” or “redeposition” of indigo onto the denim material, e.g. re-colouration of the blue threads and blue coloration of the white threads, resulting in a less contrast between the blue and white threads. In general, the higher abrasion level will lead to higher backstaining level as more dyestuff is removed and redeposited into the fabric. The process which causes high abrasion level but low backstaining level is desirable for the textile manufacture. To measure whether a process can achieve low backstaining level, the delta L* unit from one process shall be compared with a control process when both process reach the similar abrasion level (i.e. similar Delta L* unit), because the similar abrasion level general means similar amount of dyestuff removed by the process.

Abrasion is generally followed by the third step, after-treatment which generally includes washing and rinsing steps during which detergents, optical brighteners, bleaching agents or softeners may be used.

The method of the present invention of treating the textile with a GH61 polypeptide in the presence of cellulase in an aqueous solution can be applied to a biostoning process.

In one embodiment, the invention provides a method for introducing into the surface of dyed fabric or garment, localized variations in colour density in which the method comprises the step of contacting the fabric or garment with a GH61 polypetide in the presence of a cellulase. Preferably, the dyed fabric or garment is cellulosic or cellulose-containing fabric or garment. More preferably, the dyed fabric is a denim fabric, even more preferably, indigo dyed denim fabric.

In another embodiment, the invention provides a denim manufacturing process, which comprises: a) desizing of the denim fabric; b) biostoning the denim with a GH61 polypetide in the presence of a cellulase; c) rinsing.

The process of the invention may be carried out at conventional conditions in a washing machine conventionally used for stone-washing, e.g., a washer-extractor, belly washer, etc. The enzyme of the invention should be added in an effective amount.

### Enzymes

### Cellulases

In the present context, the term “cellulase” or “cellulolytic enzyme” refers to an enzyme which catalyzes the degradation of cellulose to glucose, cellobiose, triose and other cello-oligosaccharides which enzyme is understood to include a mature protein or a precursor form thereof or a functional fragment thereof, e.g., a catalytic active domain, which essentially has the activity of the full-length enzyme. Furthermore, the term “cellulolytic” enzyme is intended to include homologues or analogues of said enzyme. Suitable cellulases include those of animal, vegetable or microbial origin. Microbial origin is preferred.

The cellulolytic enzyme may be a component occurring in a cellulase system produced by a given microorganism, such a cellulase system mostly comprising several different cellulase enzyme components including those usually identified as, e.g., cellobiohydrolases (E.C. 3.2.1.91), endoglucanases (E.C. 3.2.1.4), and beta-glucosidases (E.C. 3.2.1.21).

The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

Preferably, the cellulase in the present invention is cellulase (or cellulolytic enzyme) having abrasion effect. For the purpose of the present invention, abrasion level is measured under conditions as specified in Example 1, by cellulase treatment in Launder-O-Meter (LOM) at 55° C., pH 6.5 for 2 hours, with cellulase dosage of 0.05 mg/g. In a preferred embodiment of the present invention, the cellulase having abrasion effect shows at least 0.5 Delta L* unit, preferably at least 1, more preferably at least 1.5, more preferably at least 2, more preferably at least 2.5, more preferably at least 3, more preferably at least 3.5, more preferably at least 4, more preferably at least 4.5, more preferably at least 5, more preferably at least 5.5, more preferably at least 6, even more preferably at least 6.5, and even most preferably at least 7 Delta L* unit. Preferably, the cellulase (or cellulolytic enzyme) having abrasion effect in the present invention is an endoglucanse.

Alternatively, the cellulolytic enzyme may be a single component, i.e. a component essentially free of other cellulase enzymes usually occurring in a cellulase system produced by a given microorganism, the single component typically being a recombinant component, i.e. produced by cloning of a DNA sequence encoding the single component and subsequent cell transformed with the DNA sequence and expressed in a host, for example as described e.g., International Patent Application WO 91/17243 and which is hereby incorporated by reference. The host is preferably a heterologous host, but the host may under certain conditions also be the homologous host.

The cellulase to be used according to the present invention may be any cellulase component having cellulolytic activity either in the acid, the neutral or the alkaline pH-range. Preferably, the component is a microbial endoglucanase (EC 3.2.1.4), preferably of fungal or bacterial origin, which may be derived or isolated and purified from microorganisms which are known to be capable of producing cellulolytic enzymes, e.g., species of the genera mentioned below. The derived cellulases may be either homologous or heterologous cellulases. Preferably, the cellulases are homologous. However, a heterologous component, which is derived from a specific microorganism and is immunoreactive with an antibody raised against a highly purified cellulase component possessing the desired property or properties, is also preferred. Preferably, the cellulase used in the present invention is an endoglucanase (EC 3.2.1.4).

For purposes of the present invention, endoglucanase activity is determined using carboxymethyl cellulose (CMC) as substrate according to the procedure of part VI in page 264 of Ghose, 1987, Pure and Appl. Chem. 59: 257-268.

Examples of specific endoglucanase useful according to the present invention are: cellulases derived from any of the fungal genera Acremonium, Ascobolus, Aspergillus, Chaetomium, Chaetostylum, Cladorrhinum, Colletotrichum, Coniothecium, Coprinus, Crinipellis, Cylindrocarpon, Diaporthe, Diplodia, Disporotrichum, Exidia, Fomes, Fusarium, Geotrichum, Gliocladium, Humicola, Irpex, Macrophomina, Melanocarpus, Microsphaeropsis, Myceliophthora, Nectia, Neocallimastix, Nigrospora, Nodulisporum, Panaeolus, Penicillium, Phanerochaete, Phycomyces, Piromyces, Poronia, Rhizomucor, Rhizophyctis, Saccobolus, Schizophyllum, Scytalidium, Sordaria, Spongopellis, Systaspospora, Thermomyces, Thielavia, Trametes, Trichothecium, Trichoderma, Volutella, Ulospora, Ustilago, Xylaria; especially acid cellulases derived from the fungal species Trichoderma reesei, Trichoderma viride, Trichoderma longibrachiatum; cellulases from the fungal species Ascobolus stictoideus, Aspergillus aculeatus, Chaetomium cuniculorum, Chaetomium brasiliense, Chaetomium murorum, Chaetomium virescens, Chaetostylum fresenii, Cladorrhinum foecundissimum, Colletotrichum lagenarium, Coprinus, Crinipellis scabella, Cylindrocarpon, Diaporthe syngenesia, Diplodia gossypina, Exidia glandulosa, Fomes fomentarius, Fusarium oxysporum, Fusarium poae, Fusarium solani, Fusarium anguioides, Geotrichum, Gliocladium catenulatum, Humicola nigrescens, Humicola grisea, Irpex, Macrophomina phaseolina, Melanocarpus albomyces, Microsphaeropsis, Myceliophthora thermophila, Nectria pinea, Neocallimastix patriciarum, Nigrospora, Nodulisporum, Panaeolus retirugis, Penicillium chrysogenum, Penicillium verruculosum, Phanerochaete, Phycomyces nitens, Piromyces, Poronia punctata, Rhizomucor pusillus, Rhizophlyctis rosea, Saccobolus dilutellus, Schizophyllum commune, Scytalidium thermophilum, Sordaria fimicola, Sordaria macrospora, Spongopellis, Syspastospora boninensis, Thermomyces verrucosus, Thielavia thermophila, Thielavia terrestris, Trametes sanguines, Trichothecium roseum, Trichoderma harzianum, Volutella colletotrichoides, Ulospora bilgramii, Ustilago maydis, Xylaria hypoxylon, Myceliophthora thermophila, Humicola insolens, Humicola lanuginosa, Humicola grisea; and a GH45 endoglucanase derived from Humicola insolen shaving the amino acid sequence disclosed in PCT Patent Application No. WO 91/17243, SEQ ID NO: 2, or an endoglucanase from Thielavia terrestis as described in WO 96/29397, or a variant having an amino acid sequence being at least 60%, preferably at least 70%, more preferably 75%, more preferably at least 80%, more preferably 85%, especially at least 90% identity therewith; and cellulases from the bacterial genera Bacillus, Pseudomonas, Saccharothrix, Cellvibrio, Thermomonospora; especially from the species Bacillus lentus, Bacillus agaradhaerens, Bacillus licheniformis, Pseudomonas cellulose, Saccharothrix australiensis, Saccharothrix texasensis, Saccharothrix waywayandensis, Saccharothrix cryophilis, Saccharothrix flava, Saccharothrix coeruleofusca, Saccharothrix longispora, Saccharothrix mutabilis ssp. capreolus, Saccharothrix aerocolonigenes, Saccharothrix mutabilis ssp. mutabilis, Saccharothrix syringae, Cellvibrio mixtus, Thermomonospora fusca. References are made to the detailed disclosure of the mentioned cellulases in the International Patent Applications published as WO94/01532, WO94/14953, WO96/11262, WO96/19570 and WO96/29397; further examples are the cellulases disclosed in the published EP271004.

Endoglucanases with an anti-redeposition effect may be obtained from fungal endoglucanases lacking a carbohydrate-binding module (CBM) from a number of bacterial sources. Some sources are Humicola insolens, Bacillus sp. deposited as DSM 12648, Bacillus sp. KSMS237 deposited as FERM P-16067, Panibacillus polymyxa, and Panibacillus pabuli. Specific anti-redeposition endoglucanases are disclosed in FIG. 14 of WO 91/17244 (hereby incorporated by reference), WO 04/053039 SEQ ID NO: 2 (hereby incorporated by reference), JP 2000210081 position 1 to 824 of SEQ ID NO: 1 (hereby incorporated by reference).

Examples of commercially available cellulase enzyme products useful in the method of the present invention are: Cellusoft®, Celluclast®, Denimax® Acid, Denimax® Ultra (all available from Novo Nordisk A/S, DK-2880 Bagsvaerd, Denmark); Indiage™, Primafast™ (both from Genencor International Inc., U.S.A.); Powerstone™ (from Iogen, Canada); Ecostone™, Biotouch™ (both from AB Enzymes, Finland); Rocksoft™ (from CPN, U.S.A.), and Sanko Bio™ (from Meiji/Rakuto Kasei Ltd., Japan).

### Proteases

In a preferred embodiment, proteases are used in the present invention. Suitable proteases include those of animal, vegetable or microbial origin. Microbial origin is preferred. Chemically modified or protein engineered mutants are included. The protease may for example be a metalloprotease (EC 3.4.17 or EC 3.4.24) or a serine protease (EC 3.4.21), preferably an alkaline microbial protease or a trypsin-like protease. Examples of proteases are subtilisins (EC 3.4.21.62), especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g., of porcine or bovine origin) and the Fusarium protease described in WO 89/06270 and WO 94/25583.

Preferred commercially available protease enzymes include Alcalase®, Savinase®, Primase®, Duralase®, Esperase®, and Kannase® (Novozymes NS), Maxatase®, Maxacal®, Maxapem®, Properase®, Purafect®, Purafect OxP®, FN2™, and FN3™ (Genencor International Inc.).

### Lipases

In other embodiments of the present invention, lipases are used in the present invention. Suitable lipases include those of bacterial or fungal origin. Chemically or genetically modified mutants of such lipases are included in this connection. The lipase may for example be triacylglycerol lipase (EC3.1.1.3), phospholipase A2 (EC 3.1.1.4), Lysophospholipase (EC 3.1.1.5), Monoglyceride lipase (EC 3.1.1.23), galactolipase (EC 3.1.1.26), phospholipase A1 (EC 3.1.1.32), Lipoprotein lipase (EC 3.1.1.34). Examples of useful lipases include a Humicola lanuginosa lipase, e.g., as described in EP 258 068 and EP 305 216; a Rhizomucor miehei lipase, e.g., as described in EP 238 023 or from H. insolens as described in WO 96/13580; a Candida lipase, such as a C. antarctica lipase, e.g., the C. antarctica lipase A or B described in EP 214 761; a Pseudomonas lipase, such as one of those described in EP 721 981 (e.g., a lipase obtainable from a Pseudomonas sp. SD705 strain having deposit accession number FERM BP-4772), in PCT/JP96/00426, in PCT/JP96/00454 (e.g., a P. solanacearum lipase), in EP 571 982 or in WO 95/14783 (e.g., a P. mendocina lipase), a P. alcaligenes or P. pseudoalcaligenes lipase, e.g., as described in EP 218 272, a P. cepacia lipase, e.g., as described in EP 331 376, a P. stutzeri lipase, e.g., as disclosed in GB 1,372,034, or a P. fluorescens lipase; a Bacillus lipase, e.g., a B. subtilis lipase (Dartois et al. (1993) Biochemica et Biophysica Acta 1131:253-260), a B. stearothermophilus lipase (JP 64/744992) and a B. pumilus lipase (WO 91/16422).

Suitable commercially available lipases include Lipex®, Lipolase® and Lipolase Ultra®, Lipolex®, Lipoclean® (available from Novozymes NS), M1 Lipase™ and Lipomax™ (available from Genencor Inc.) and Lipase P “Amano” (available from Amano Pharmaceutical Co. Ltd.). Commercially available cutinases include Lumafast™ from Genencor Inc.

### Cutinases

In other embodiments, cutinases are used in the present invention. Potentially useful types of lipolytic enzymes include cutinases (EC 3.1.1.74), e.g., a cutinase derived from Pseudomonas mendocina as described in WO 88/09367, or a cutinase derived from Fusarium solani pisi (described, e.g., in WO 90/09446). Due to the lipolytic activity of cutinases they may be effective against the same stains as lipases. Commercially available cutinases include Lumafast™ from Genencor Inc.

### Amylases

In other embodiments, amylases are used in the present invention. Amylases comprise e.g., alpha-amylases (EC 3.2.1.1), beta-amylases (EC 3.2.1.2) and/or glucoamylases (EC 3.2.1.3) of bacterial or fungal origin. Chemically or genetically modified mutants of such amylases are included in this connection. Alpha-amylases are preferred in relation to the present invention. Relevant alpha-amylases include, for example, α-amylases obtainable from Bacillus species, in particular a special strain of B. licheniformis, described in more detail in GB 1296839.

Further examples of useful amylases are the alpha-amylases derived from Bacillus sp.; the alpha-amylases shown in SEQ ID NO 1 and 2 of WO 95/26397 (hereby incorporated by reference); the AA560 alpha-amylase derived from Bacillus sp. DSM 12649 disclosed as SEQ ID NO: 2 in WO 00/60060 (hereby incorporated by reference) and the variants of the AA560 alpha-amylase, including the AA560 variant disclosed in Example 7 and 8 (hereby incorporated by reference).

Relevant commercially available amylases include Natalase®, Stainzyme®, Duramyl®, Termamyl®, Termamyl™ Ultra, Fungamyl® and BAN® (all available from Novozymes A/S, Bagsvaerd, Denmark), and Rapidase® and Maxamyl® P (available from DSM, Holland) and Purastar®, Purastar OxAm and Powerase™ (available from Danisco A/S).

Other useful amylases are CGTases (cyclodextrin glucanotransferases, EC 2.4.1.19), e.g., those obtainable from species of Bacillus, Thermoanaerobactor or Thermoanaerobacterium.

### Hemicellulases

In other embodiments, hemicellulases are used in the present invention. Hemicelluloses are the most complex group of non-starch polysaccharides in the plant cell wall. They consist of polymers of xylose, arabinose, galactose, mannose and/or glucose which are often highly branched and connected to other cell wall structures. Hemicellulases of the present invention therefore include enzymes with xylanolytiactivity, arabinolytic activity, galactolytic activity and/or mannolytic activity. The hemi-cellulases of the present invention may for example be selected from xylanases (EC 3.2.1.8, EC 3.2.1.32, and EC 3.2.1.136), xyloglucanases (EC 3.2.1.4 and EC 3.2.1.151), arabinofuranosidases (EC 3.2.1.55), acetylxylan esterases (EC EC 3.1.1.72), glucuronidases (EC 3.2.1.31, EC 3.2.1.56, 3.2.1.128 and 3.2.1.139), glucanohydrolase (EC 3.2.1.11, EC 3.2.1.83 and EC 3.2.1.73), ferulic acid esterases (EC 3.1.1.73), coumaric acid esterases (EC 3.1.1.73), mannanases (EC 3.2.1.25; EC 3.2.1.78 and EC 3.2.1.101), arabinosidase (EC 3.2.1.88), arabinanases (EC 3.2.1.99), galactanases (EC 3.2.1.89, EC 3.2.1.23 and 3.2.1.164) and lichenases (EC 3.2.1.73). This is, however, not to be considered as an exhausting list.

Mannanase is a preferred hemicellulase in relation to the present invention. Mannanases hydrolyse the biopolymers made up of galactomannans. Mannan containing stains often comprise guar gum and locust bean gum, which are widely used as stabilizers in food and cosmetic products. Suitable mannanases include those of bacterial or fungal origin. Chemically or genetically modified mutants are included. In a preferred embodiment the mannanase is derived from a strain of the genus Bacillus, especially Bacillus sp. 1633 disclosed in positions 31-330 of SEQ ID NO:2 or in SEQ ID NO: 5 of WO 99/64619 (hereby incorporated by reference) or Bacillus agaradhaerens, for example from the type strain DSM 8721. A suitable commercially available mannanase is Mannaway® produced by Novozymes A/S or Purabrite™ produced by Genencor a Danisco division.

Xylanase is a preferred hemicellulase in relation to the present invention. A suitable commercially available xylanase is Pulpzyme® HC (available from Novozymes A/S).

### Pectinases

In other embodiments, pectinases are used in the present invention. The term pectinase or pectolytic enzyme is intended to include any pectinase enzyme defined according to the art where pectinases are a group of enzymes that catalyze the cleavage of glycosidic linkages. Basically three types of pectolytic enzymes exist: pectinesterase, which only removes methoxyl residues from pectin, a range of depolymerizing enzymes, and protopectinase, which solubilizes protopectin to form pectin (Sakai et al., (1993) Advances in Applied Microbiology vol 39 pp 213-294). Example of a pectinases or pectolytic enzyme useful in the invention is pectate lyase (EC 4.2.2.2 and EC 4.2.2.9), polygalacturonase (EC 3.2.1.15 and EC 3.2.1.67), polymethyl galacturonase, pectin lyase (EC 4.2.2.10), galactanases (EC 3.2.1.89), arabinanases (EC 3.2.1.99) and/or pectin esterases (EC 3.1.1.11).

Suitable pectinolytic enzymes include those described in WO 99/27083, WO 99/27084, WO 00/55309 and WO 02/092741.

Suitable pectate lyases include those of bacterial or fungal origin. Chemically or genetically modified mutants are included. In a preferred embodiment the pectate lyase is derived from a strain of the genus Bacillus, especially a strain of Bacillus substilis, especially Bacillus subtilis DSM14218 disclosed in SEQ ID NO:2 or a variant thereof disclosed in Example 6 of WO 02/092741 (hereby incorporated by reference) or a variant disclosed in WO 03/095638 (hereby incorporated by reference). Alternatively the pectate lyase is derived from a strain of Bacillus licheniformis, especially the pectate lyases disclosed as SEQ ID NO: 8 in WO 99/27083 (hereby incorporated by reference) or variants thereof as described in WO 02/06442.

Suitable commercially available pectate lyases are Pectaway® or X Pect® produced by Novozymes A/S.

### Textile Composition

The present invention also encompasses textile composition comprising a GH61 polypeptide and a cellulase.

The textile composition may be adapted for specific uses, such as biostoning or biopolishing. The use of a GH61 polypeptide together with a cellulase can provide improved textile performance such as increasing the denim abrasion level, reducing backstaining level, promoting the dye released from the textile, colour clarification and reduction of pilling.

In the present invention, GH61 polypeptide enhances the cellulase activity by reducing the amount of cellulase required to reach the same degree of abrasion or depilling.

In some embodiments of the invention, the composition containing a GH61 polypeptide and a cellulase further comprises other components, including without limitation other enzymes, as well as one or more of surfactants, bleaching agents, antifoaming agents, builder systems, and the like, that enhance the biopolishing and/or biostoning process and/or provide superior effects related to, e.g., dyeability and/or wettability.

Enzymes suitable for use in the present invention include without limitation proteases, lipases, cutinases, amylases, pectinases, hemicellulases, oxidoreductases, peroxidases, laccases, and transferases.

In one embodiment, the textile composition comprises one or more of the GH 61 polypeptides selected from the group consisting of an amino acid sequence that has a degree of identity to the mature polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, or SEQ ID NO: 32 of at least 60%, e.g., at least 65%, at least 70%, at least 75%, at least 80%, at least 85%, at least 90%, at least 91%, at least 92%, at least 93%, at least 94%, or at least 95%, at least 96%, at least 97%, at least 98%, at least 99%, or at least 100%.

In an even more preferred aspect, the textile composition further comprises a cosubstance, such as cysteine.

The textile composition can be in any form, such as a solid, liquid, paste, gel or any combination thereof.

### Process Conditions

GH61 polypeptides in combination with cellulases can be used during textile manufacturing process, especially during a biostoning or a biopolishing process.

It is advised that a suitable liquor/textile ratio to be used in the present method may be in the range of from about 20:1 to about 1:1, preferably in the range of from about 15:1 to about 3:1, more preferably in the range of from 15:1 to 5:1 (Volumn/weight, ml/mg).

In conventional “biostoning” or “biopolishing” processes, the reaction time is usually in the range of from about 10 minutes to about 8 hours. Preferably the reaction time is within the range of from about 20 minutes to about 180 minutes, more preferably the reaction time is within the range of from about 30 minutes to about 150 minutes, most preferably the reaction time is within the range of from about 45 minutes to about 120 minutes.

The pH of the reaction medium greatly depends on the enzyme(s) in question. Preferably the process of the invention is carried out at a pH in the range of from about pH 3 to about pH 11, preferably in the range of from about pH 4 to about pH 8, or within the range of from about pH 4.5 to about pH 7.5.

The process of the present invention is able to function at a temperature below 90° C., preferably below 75° C., more preferably below 65° C., more preferably below 50° C., more preferably below 40° C., even more preferably below 30° C.

In some embodiments, the process of the present invention is conducted at the temperature range of 5-90° C., preferably 10-90° C., preferably 10-80° C., more preferably 10-75° C., more preferably 15-65° C., more preferably 20-65° C., more preferably 30-65° C., and even more preferably 30-55° C.

Enzyme dosage greatly depends on the enzyme reaction time, i.e. a relatively short enzymatic reaction time necessitates a relatively increased enzyme dosage, and vice versa. In general, enzyme dosage may be stipulated in accordance with the reaction time available.

The amount of GH61 polypeptide to be used according to the method of the present invention depends on many factors, but according to the invention the concentration of the of GH61 polypeptide in the aqueous medium may be from about 0.001 to about 10 milligram enzyme protein per gram of fabric, preferably 0.02-5 milligram of enzyme protein per gram (g) of fabric, preferably 0.05-2 milligram of enzyme protein per gram of fabric, more preferably 0.04-0.6 milligram of enzyme protein per gram of fabric.

The amount of cellulase (or cellulolytic enzyme) to be used according to the method of the present invention depends on many factors, but according to the invention the concentration of the cellulolytic enzyme in the aqueous medium may be from about 0.001 to about 10 milligram (mg) enzyme protein per g of fabric, preferably 0.02-5 milligram of enzyme protein per gram of fabric, more preferably 0.05-2 milligram of enzyme protein per gram of fabric.

According to the invention the concentration of the cosubstance, such as L-cystein in the aqueous medium may be preferably 0.1-50 mM, more preferably 0.5-25 mM, more preferably 1-10 mM, even more preferably 4-8 mM.

The aqueous composition used in the method of the invention may further comprise one or more enzymes selected from the group consisting of proteases, lipases, cutinases, cellulases, hemicellulases, pectinases, amylases, oxidoreductases, peroxidases, laccases, and transferases.

The process of the present invention can provide the effect of increased abrasion level, and/or low backstaining level as compared to a textile composition without the treatment of the glycosyl hydrolase family 61 polypeptide. The process of the present invention can also enhance the dye release from the fabric, which will give the fabric a different style after treatment.

### EXAMPLES

### Materials & Methods

Cellusoft Neupolish 8000 L® (a Thielavia terrestis mono-component endoglucanase product commercially available from Novozymes A/S)
Carezyme 4500T® (a mono-component Humicola insolens GH45 endoglucanase product, commercially available from Novozymes A/S)
Cellusoft L® (a Trichoderma reesei multi-component cellulase product, commercially available from Novozymes A/S)
Mature polypeptide of Ta GH61: Thermoascus aurantiacus GH61A polypeptide shown as amino acids 22 to 249 of SEQ ID NO:1 (described in WO 2005/074656)
Mature polypeptide of Af GH61: Aspergillus fumigatus GH61B polypeptide shown as amino acids of 22 to 250 SEQ ID NO:2 (described in US 2010124769)
Mature polypeptide of Ts GH61: Talaromyces stipitatus GH61 polypeptide shown as amino acids of 22 to 320 SEQ ID NO:33 (UNIPROT: B8M2G3)

### Colour Measurement

The abrasion level and backstaining level of the denim samples were determined by measuring the reflectance with pre-calibrated DataColor SF450X, alternatively an equivalent apperatus can be used. Four readings were taken for each sample, and the average of the readings were used. The abrasion level was evaluated with the index CIE L* on the blue side (front side) of the sample, and the backstaining level was evaluated with the index CIE b* on the back side of the sample.

L* indicates the change in white/black on a scale from 0 to 100, and a decrease in L* means an increase in black colour (decrease in white colour) and an increase in L* means an increase in white colour (decrease in black colour). Delta L* unit=L* of the swatch treated with a certain cellulase−L* of the swatch before cellulase treatment. The larger the Delta L* unit is the higher is the denim abrasion level, e.g. a Delta L* unit of 4 has higher abrasion level than Delta L* unit of 3.

b* indicates the change in blue/yellow, and a decrease in b* means an increase in blue colour (decrease in yellow colour), and an increase in b* means an increase in yellow colour (decrease in blue colour). Delta b* units=b* of the swatch treated with a certain cellulase−b* of the swatch before cellulase treatment. A larger Delta b* unit corresponds to a lower backstaining level, e.g. a Delta b* unit of −1.5 has lower backstaining level than the Delta b* unit of −2.5.

### Dye Release

The dye release capacity was determined with pre-calibrated Spectrophotometer UV 1700. The treating bath from each beaker was collected and centrifuged at 4000 rpm for 15 min, to further collect the supernatant for absorption assay at 590 nm. The higher OD590 values mean more dye is released from the fabric into the solutions, which will give the fabric a new finishing style.

### Protein Content

The enzyme protein in an enzyme product can be measured with BCA™ Protein Assay Kit (product number 23225, commercial available from Thermo Fisher Scientific Inc.) according to the product manual.

### Example 1

### Denim Abrasion with Cellulase and Ta GH61 in Launder-O-Meter

The effects of mature polypeptide of Ta GH61 on the denim abrasion by Cellusoft Neupolish 8000 L® were tested in Launder-O-Meter (SDL-Atlas LP2), including the abrasion, backstaining and the color of the treating bath.

Raw denim was desized and cut to 12.5 cm tall and 23 cm long. The denim was cut and sewn, forming a tube with height of 12.5 cm and weight of about 14 g. The tubes were placed in a conditioned room (65% relative humidity, 20° C.) for 24 hours before they were numbered, weighed by the analytical balance and recorded. One conditioned tube was placed in each 500 ml beaker, with the blue side facing inward. For each beaker, 30 big nuts (M10 M6M-SR-A4-80, acid proof), 10 small nuts (M6 M6M-SR-A4-80, acid proof), 7 big star magnets (diam. 17 mm, item no. 3-CO-411117, Cowie, Schweiz via Bie & Berntsen), and 3 small star magnets (diam. 14 mm, item no. 3-CO-11117, Cowie, Schweiz via Bie & Berntsen) were used to supply the mechanical aids. Then the buffer (50 mM phosphate buffer, pH=6.5) and the enzyme solutions were added according to Table 1, based on the calculation of actual fabric weights, to make a total volume around 50 ml, which would create a liquid to fabric ratio of 3.8:1(v/w).

The Launder-O-Meter (LOM) machine was started after the required program was chosen, and it would hold when the temperature reached 55° C. Each beaker was fitted with a lid lined with 2 neoprin gaskets and close tightly with the metal clamping device. The beakers were loaded into the preheated LOM. Metal racks were used to accommodate and secure 6 beakers, in the horizontal position, in each of the 4 drum positions. The LOM lid was closed and the washing program was continued and the timing was initiated. 2 hours later, all beakers were removed and the denim samples were transferred to the inactivation solution (2 g/L sodium carbonate) at 85° C. for 10 minutes. Then the swatches were rinsed in hot water for 2 times and in cold water for 2 times. The denim samples were tumble-dried (AEG, LAVATHERM 37700, Germany), and then conditioned for 24 hours at 20° C., 65% relative humidity prior to evaluation.

The treating bath from each beaker was also collected and centrifuged at 4000 rpm for 15 min, to further collect the supernatant for absorption assay at 590 nm by Spectrophotometer UV 1700.

The abrasion and backstaining level of the denim samples were determined by measuring the reflectance with pre-calibrated DataColor SF450X. Four readings were taken for each sample. The abrasion level was evaluated with the index CIE L* of the blue side of the sample, and the backstaining level was evaluated with the index CIE b* of the back side of the sample. For both L* and b*, 4 readings were conducted for each fabric and the average of the four readings was used.

Cellulase from Cellusoft Neupolish 8000 L® was measure by BCA™ Protein Assay Kit. As shown in Table 1, together with cellulase from Cellusoft Neupolish 8000 L® of 0.05 mg enzyme protein/g fabric, the addition of 0.042 or 0.672 mg Ta GH61/g fabric increased the abrasion level from 7.11 to 8.73 or 8.14 represented by the delta L* on the fabric face, while retaining or even slightly decreasing the backstaining level from −3.58 to −3.41 or −3.21 represented by the delta b* on the fabric back. Another remarkable effect was, with the addition of Ta GH61, the color of the treating bath became significantly darker. And the boosting effect in bath color was in line with the dosage of Ta GH61. A synergy effect in increasing the bath color was found between cellulase and Ta GH61 during denim abrasion with higher dosage of GH61 used together with cellulase giving OD590 result of 0.62.

TABLE 1
Results of Denim abrasion in LOM at 55° C., pH 6.5, 2 hours

| | Cellulase (mg | GH61(mg | Denim fabrics | | Bath |
|---|---|---|---|---|---|
| | enzyme protein/ | protein/ | Delta | Delta | OD |
| Enzyme used | g fabric) | g fabric) | L* | b* | 590 |
| Cellulase | 0.05 | 0 | 7.11 | −3.58 | 0.19 |
| | 0.1 | 0 | 9.17 | −3.59 | 0.27 |
| Cellulase + | 0.05 | 0.042 | 8.73 | −3.41 | 0.29 |
| Ta GH61 | 0.05 | 0.672 | 8.14 | −3.21 | 0.62 |
| Ta GH61 | 0 | 0.672 | 0.84 | −1.15 | 0.25 |

Note:
average of triple samples for each enzyme combination.

### Example 2

### Denim Abrasion with Cellulase and Additional Af GH61 in LOM

The effects of the mature polypeptide of Af GH61 on denim abrasion of Cellusoft Neupolish 8000 L® were also tested with the protocol same as Example 1. The treating time here was 2 hours.

As shown in Table 2, together with cellulase from Cellusoft Neupolish 8000 L® of 0.016 mg enzyme protein/g fabric, the addition of 0.032 mg Af GH61/g fabric was found to boost the abrasion, which was represented by the increase of delta L* from 7.1 to 8.1 with the addition of Af GH61. And Af GH61 also made the bath more bluish, indicating an increased dye release from the fabric into the supernatent, which was represented by the higher OD 590 value in the solution.

TABLE 2
Results of Denim abrasion in LOM at 55° C., pH 6.5, 2 hours

| | Cellulase (mg | GH61 (mg | Denim fabrics | | Bath |
|---|---|---|---|---|---|
| | enzyme protein/ | protein/ | Delta | Delta | OD |
| Enzyme used | g fabric) | g fabric) | L* | b* | 590 |
| Blank | 0 | 0 | 1.9 | −1.1 | 0.11 |
| Cellulase | 0.016 | 0 | 7.1 | −3.1 | 0.13 |
| Cellulase + | 0.016 | 0.032 | 8.1 | −3.4 | 0.32 |
| Af GH61 | | | | | |
| Af GH61 | 0 | 0.032 | 2.6 | −1.3 | 0.26 |

Note:
average of triple samples for each enzyme combination.

### Example 3

### Denim abrasion with Cellulase, Ta GH61 and L-cysteine in LOM

The effects of L-cysteine on denim abrasion by Cellusoft Neupolish 8000 L®, and Cellusoft Neupolish 8000 L®/the mature polypeptide of Ta GH61 mixture were tested in LOM. The trial conditions were the same as Example 1, except 0-20 mM L-cysteine was added together with Cellusoft Neupolish 8000 L® or the Cellusoft Neupolish 8000 L®/Ta GH61 mixture. The treating time in this example was set as 0.5 h.

As shown in Table 3, it was confirmed that the addition of 0.05 mg Ta GH61/g fabric together with cellulase from Cellusoft Neupolish 8000 L® of 0.05 mg enzyme protein/g fabric could improve the abrasion from 1.14 to 1.59, while decreasing the backstaining level from −1.54 to −1.36. The bath also became more bluish with the addition of Ta GH61. And further addition of 5 mM L-cysteine as a cosubstance in the Cellusoft Neupolish 8000 L®/Ta GH61 mixture could boost the abrasion to a higher level from 1.59 to 1.80 and reduce the backstaining from −1.36 to −1.26. 5 mM was found to be a suitable concentration for L-cysteine as the booster to the mixture to deliver higher abrasion level and more bluish bath. When the concentration was further increased to 10 or 20 mM, L-cysteine lost its boosting effects or even became an inhibitor to the mixture.

TABLE 3
Results of Denim abrasion in LOM at 55° C., pH 6.5, 0.5 hours

| | Cellulase | | | | | |
|---|---|---|---|---|---|---|
| | (mg enzyme | GH61 | | | | Bath |
| | protein/g | (mg protein/g | L-cysteine | Denim fabrics | | OD |
| Enzyme used | fabric) | fabric) | (mM) | Delta L* | Delta b* | 590 |
| Cellulase | 0.05 | 0 | 0 | 1.14 | −1.54 | 0.09 |
| | 0.1 | 0 | 0 | 1.79 | −1.70 | 0.12 |
| Cellulase + Ta GH61 | 0.05 | 0.05 | 0 | 1.59 | −1.36 | 0.15 |
| Cellulase + Ta GH61 + | 0.05 | 0.05 | 5 | 1.80 | −1.26 | 0.16 |
| L-cysteine | | | | | | |
| Cellulase + Ta GH61 + | 0.05 | 0.05 | 10 | 1.48 | −1.34 | 0.14 |
| L-cysteine | | | | | | |
| Cellulase + Ta GH61 + | 0.05 | 0.05 | 20 | 0.92 | −1.60 | 0.04 |
| L-cysteine | | | | | | |

Note:
average of triple samples for each enzyme combination.

### Example 4

### Denim Abrasion with Cellulase and Ta GH61 in Wascator

Denim abrasion trials were conducted in a wascator (Electrolux, Switzerland). For each trial, five pieces and two types of denim tubes plus a small piece of denim filler, which weighed up around 1 kg, were loaded together. 1.0 g/L sodium acetate and acetate were used to control the bath at pH 6-7. Datacolor SF450 was used to evaluate the abrasion level with the index CIE L* of the blue side of the sample, and to evaluate the backstaining level with the index CIE b* of the back of the sample. For both L* and b*, 8 readings were conducted for each fabric. Visual inspection was also applied for the washing pattern comparison. The trials conditions were described as below:

| | |
|---|---|
| Pre-wash | 25° C., 5 min; liquid to fabric ratio 15:1 (w/w) |
| Drain | |
| Main wash | 55° C., 60 min; liquid to fabric ratio 15:1 (w/w); |
| | pH 6-6.2 with 1.0 g/L NaAc and adjusted with HAc, |
| | enzyme solutions were added according to Table 4 |
| Drain | |
| Rinse | 25° C., 5 min; liquid to fabric ratio 20:1 (w/w) |
| Drain | |
| Rinse | 25° C., 5 min; liquid to fabric ratio 20:1 (w/w) |
| Drain | |
| Extracted and | |
| Tumble-dried | |

As shown in Table 4, the addition of 82.5 mg Ta GH61 together with cellulase from Cellusoft Neupolish 8000 L® or cellulase from Carezyme 4500T® significantly boosted the abrasion levels and reduced the backstaining level. For Cellusoft Neupolish 8000 L®, the addition of Ta GH61 increased the denim face L* by 1.52 but just slightly increased the denim backstaining b* by 0.19. For Carezyme 4500T, the addition of Ta GH61 simultaneously increased the abrasion by 0.74 L* and reduced back backstaining b* by 0.09. And visual inspection confirmed the abrasion boosting and backstaining reduction effects of Ta GH61 on both cellulases.

TABLE 4
Results of Denim abrasion in wascator at 55° C., pH 6-6.5, 2 hours

| | Cellulase (mg | GH61(mg | | |
|---|---|---|---|---|
| | enzyme protein/ | protein/ | Denim fabrics | |
| Enzyme used | g fabric) | g fabric) | L* | b* |
| Cellulse of Cellusoft | 0.04 | 0 | 21.82 | −9.72 |
| Neupolish 8000 L | | | | |
| Cellulase of Cellusoft | 0.04 | 0.08 | 23.34 | −9.91 |
| Neupolish 8000 L + Ta | | | | |
| GH61 | | | | |
| Cellulase of Carezyme | 0.04 | 0 | 21.74 | −9.72 |
| 4500T | | | | |
| Cellulase of Carezyme | 0.04 | 0.08 | 22.48 | −9.63 |
| 4500T + Ta GH61 | | | | |

Notes:
average of duplicate samples for each enzyme combination.

### Example 5

### Denim Abrasion with Cellulase and Additional Ts GH61 in LOM

Ts GH61 (Talaromyces stipitatus GH61) was tested with Cellusoft Neupolish 8000 L® under the same protocol as Example 1. The treating time was 2 hours.

As shown in Table 5, using cellulase from Cellusoft Neupolish 8000 L® of 0.016 mg enzyme protein/g fabric, the addition of 0.032 mg Ts GH61/g fabric was found to boost the abrasion level.

TABLE 5
Results of Denim abrasion in LOM at 55° C., pH 6.5, 2 hours

| | Cellulase (mg | GH61 (mg | Denim fabrics | |
|---|---|---|---|---|
| | enzyme protein/ | protein/ | Delta | Delta |
| Enzyme used | g fabric) | g fabric) | L* | b* |
| Blank | 0 | 0 | 1.4 | −2.0 |
| Cellulase | 0.016 | 0 | 6.6 | −4.3 |
| Cellulase + Ts GH61 | 0.016 | 0.032 | 7.5 | −4.4 |
| Ts GH61 | 0 | 0.032 | 1.9 | −1.8 |

Note:
average of triple samples for each enzyme combination.

As shown in Table 5, compared with cellulase alone, Ts GH61 in combination with cellulase increased the abrasion level from 6.6 to 7.5 by the delta L* on the fabric face.

### Example 6

### Denim Abrasion with Cellulase and Additional Ta GH61a in LOM

The effects of Ta GH61 on the denim abrasion performance together with Cellusoft L® (multi-component cellulases product), was tested under the same protocol as Example 1. The treating time was 2 hours.

As shown in Table 6, using cellulase from Cellusoft L® of 0.8 mg enzyme protein/g fabric, the addition of 0.032 or 0.128 mg Ta GH61a/g fabric was found to boost the abrasion and reduce the backstaining for Cellusoft L.

TABLE 6
Results of Denim abrasion in LOM at 55° C., pH 5, 2 hours

| | Cellulase (mg | GH61 (mg | Denim fabrics | |
|---|---|---|---|---|
| | enzyme protein/ | protein/ | Delta | Delta |
| Enzyme used | g fabric) | g fabric) | L* | b* |
| Blank | 0 | 0 | 2.0 | −1.5 |
| Cellulase | 0.8 | 0 | 7.8 | −3.8 |
| | 1.2 | 0 | 11.1 | −4.0 |
| Ta GH61a | 0 | 0.128 | 1.9 | −1.9 |
| Cellulase + | 0.8 | 0.032 | 8.1 | −3.0 |
| Ta GH61 | 0.8 | 0.128 | 8.7 | −3.5 |

Note:
average of triple samples for each enzyme combination.

As shown in Table 6, compared with using 0.8 mg cellulase/g of fabric alone, the addition of 0.032 or 0.128 mg Ta GH61/g fabric with cellulase increased the abrasion level from 7.8 to 8.1 and 8.7 represented by the delta L* on the fabric face, respectively. Further, the addition of Ta GH61a could reduce the backstaining level on the back side of the denim, represented by the delta b* up from −3.8 to −3.0 and −3.5 when the Ta GH61a dosage was 0, 0.032, 0.128 mg/g, respectively.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method for treating textile with a glycosyl hydrolase family 61 polypeptide in the presence of a cellulase in an aqueous solution.

2. The method according to claim 1 wherein the method is applied in a biostoning process.

3. The method according to claim 1, wherein the textile is dyed cellulosic or cellulose-containing fabric, preferably denim fabric, more preferably indigo dyed denim fabric.

4. The method according to claim 1, wherein the method is applied in a biopolishing process.

5. The method according to claim 4, wherein the textile is yarn, fabric or garment.

6. The method according to claim 1, wherein the cellulase is an endoglucanase (EC 3.2.1.4).

7. The method according to claim 1, wherein the aqueous solution further comprises one or more enzymes selected from the group consisting of proteases, lipases, cutinases, amylases, pectinases, hemicellulases, oxidoreductases, peroxidases, laccases, and transferases.

8. The method according to claim 1, wherein a cosubstance is used together with a glycosyl hydrolase family 61; preferably the cosubstance is cysteine.

9. The method according to claim 1, wherein the glycosyl hydrolase family 61 polypeptide is applied in the range of from 0.001 to about 10 milligram enzyme protein per gram of fabric.

10. The method according to (Original), wherein the cellulase is applied in the range from 0.001 to about 10 milligram enzyme protein per gram of fabric.

11. The method according to claim 1, wherein the method is conducted in the pH range of from about pH 3 to about pH 11.

12. The method according to claim 1, wherein the method is conducted in the temperature range of 10-90° C.

13. The method according to claim 1, wherein the method is conducted for 10 minutes to 8 hours.

14. The method according to claim 1, wherein the cosubstance is applied in the range of 0.1-50 mM.

15. The method according to claim 1, wherein treating the textile is manufacturing the textile.

16. The textile composition comprising a glycosyl hydrolase family 61 polypeptide and a cellulase.

17. The textile composition according to claim 16, wherein the cellulase is an endoglucanase.

18. The textile composition according to claim 16, wherein the composition further comprises one or more enzymes selected from the group consisting of proteases, lipases, cutinases, amylases, pectinases, hemicellulases, oxidoreductases, peroxidases, laccases, and transferases.

19. The textile composition according to claim 16, wherein the composition further comprises a cosubstance; preferably the cosubstance is cysteine.

20. The textile composition according to claim 16, wherein the composition further comprises a surfactant.
